(19) United States
(12) Patent Application Publication
(10) Pub. No.: US 2004/0254490 A1
(43) Pub. Date: Dec. 16, 2004

(54) Device for measuring the respiratory rate

(76) Inventor: Wendelin Egli, Seuzach (CH)

Correspondence Address: JENKENS & GILCHRIST, PC, 1445 ROSS AVENUE, SUITE 3200, DALLAS, TX, 75202, US

(21) Appl. No.: 10/483,244

(22) PCT Filed: Dec. 2, 2002
(86) PCT No.: PCT/CH02/00648

(30) Foreign Application Priority Data
Dec. 20, 2001 (CH) 2324/01

(51) Int. Cl. A62B 7/00; A61M 16/00; F16K 31/02; A61B 5/08

(52) U.S. Cl. 600/529; 128/204.21

(57) ABSTRACT

On an anti-blackout suit (1) operating according to the hydrostatic principle, a pressure measurement cell (3) is arranged at, for example, the lowest point of a liquid-conveying vein (2) which, by its internal pressure, generates the circulation stress required for the anti-blackout suit (1). Pressure changes arise as a result of the volume changes, during respiration, in the person wearing the anti-blackout suit (1), and these pressure changes are measured by the pressure measurement cell (3) and, for example, transmitted to an evaluation apparatus via a cable (5). Both a display device and also a memory device can be linked to the evaluation unit.

The present invention relates to a device for measuring the respiration rate and the breathing pattern of, for example, a person wearing an anti-blackout suit operating according to the hydrostatic principle, in accordance with the preamble of patent claim 1.

A number of devices are known for determining the physiological data of pilots, athletes or, for example, orthostasis patients, such data including pulse, blood oxygen content and respiration rate. In general, these are developments or special designs of measurement apparatuses as are used in medicine, in particular in sports medicine.

An almost universal feature of such measurement devices is that a suitable sensor has to be placed on the test person, which causes a certain degree of inconvenience or can result in a deterioration in the test person's subjective sense of well-being. There is therefore a risk of reduced acceptance of such measurement devices, or even the creation of artefacts: errors on the part of the test person caused by the existence of the measurement device.

The object of the present invention is to make available such a device for measuring respiration rate which can be put to use in the test person's usual environment with minimum effort, can be produced and installed/applied inexpensively, and provides reliable results under difficult physical and physiological conditions.

The main features of the solution to the object are set out in the characterizing part of patent claim 1, and further advantageous embodiments are set out in the subsequent claims.

The invention is explained in more detail with reference to the attached drawing, in which:

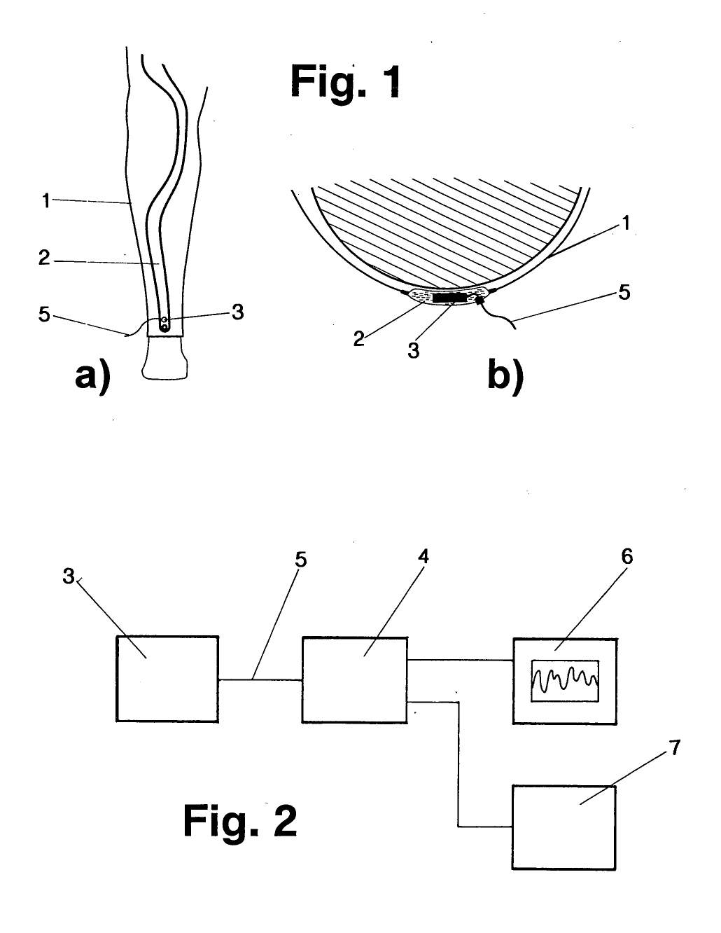

FIG. 1a shows the device according to the invention in a schematic representation,

FIG. 1b shows the arrangement from FIG. 1a in cross section,

FIG. 2 shows a block diagram,

FIG. 3 shows a first pressure/time diagram,

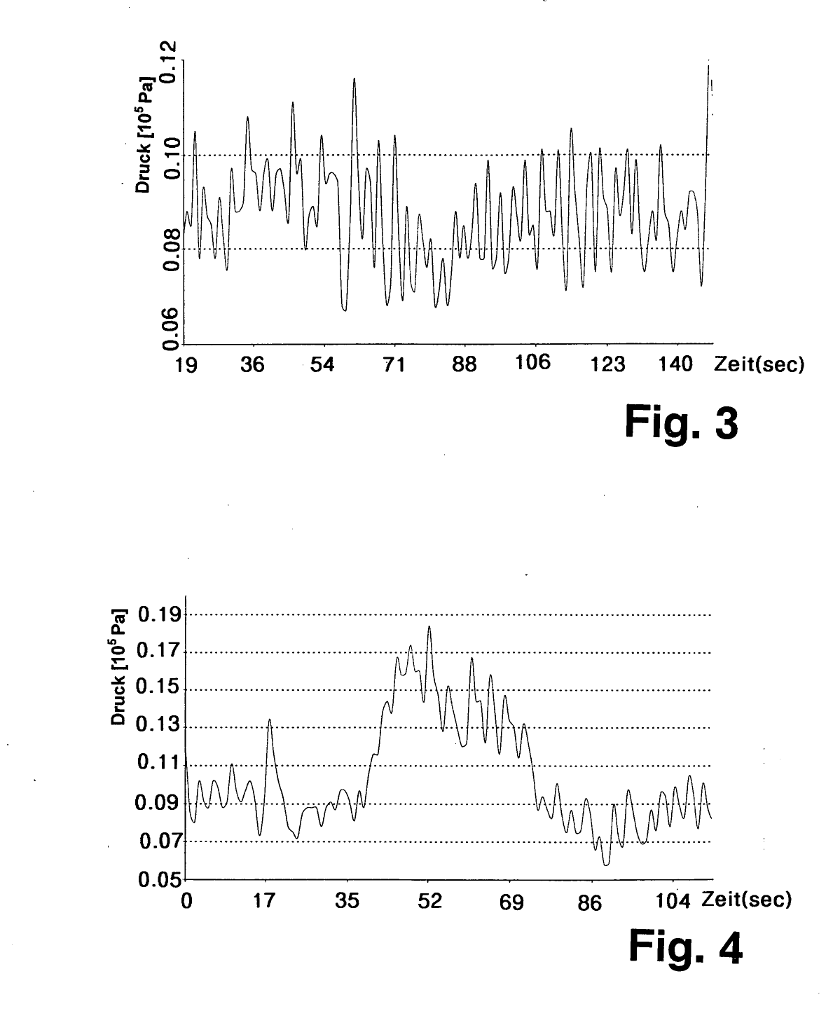

FIG. 4 shows a second pressure/time diagram.

FIGS. 1a and 1b are schematic representations of the arrangement according to the invention for use in an anti-blackout suit, an orthostasis suit or what is called a hypoxia garment. FIG. 1a shows the arrangement in a plan view from in front, and FIG. 1b in a cross section. An anti-blackout suit 1 operating in accordance with the hydrostatic principle (and hereinafter referred to as the suit), for example according to EP 0 983 190, has liquid-filled veins 2 which are worked into the suit 1 and extend in the longitudinal direction of the limbs of the person wearing this suit 1. A pressure measurement cell 3 is fitted for example at the lowest possible point of one of the veins 2, generally above the foot, in such a way that it is completely surrounded by the liquid filling the vein 2. The pressure-measurement cell 3 is connected in a suitable manner on a multicore cable 5 to an evaluation apparatus 4 shown in FIG. 2. The cable 5 can either be introduced into the vein 2 through a pressure-tight passage or connected to a pressure-tight plug. The inventive concept also encompasses signal transmission from the vein to the outside by means of an optocoupler or by radio, as is generally the case in telemetry tasks, especially in those in biomechanics.

The pressure measurement cell 3 is known per se and is, for example, of the self-calibrating type. Moreover, it is also entirely possible for a vessel containing the pressure measurement cell 3 to be connected to the vein 2, for example via a tube, in which case the pressure measurement cell 3 is connected to the cable 5 in the described manner. The pressure measurement cell 3 is therefore in liquid-communicating and pressure-communicating connection with one of the veins 2. FIG. 2 shows the block diagram of the device according to the invention. The pressure measurement cell 3 is connected via the cable 5 to the evaluation apparatus 4. The latter processes the pressure measurement values in digital form, taking into account the calibration values of the pressure measurement cell 3. These processed measurement values can either be viewed directly on a display device 6 in time sequence or can be fed to a memory device 7 for storage. Such a memory device can be set up for storing other personal parameters, for example pulse, oximetry data, ECG, EOG.

When using said suit 1, it is important that its fit is checked before the flight. Since the basic material of the suit consists of low-stretch fabric, for example aramid fibers, the quality of the fit depends on the instantaneous physical circumstances of the person wearing the suit 1. Only when the fit is tight enough can the suit 1 properly perform its task, namely that of preventing blood from flowing down into the abdominal region and legs. If the suit has been correctly fitted, a pressure diagram according to FIG. 3 is obtained. This shows a pressure/time diagram recorded with the device according to the invention during straight-line flight of a fighter aircraft.

Superposed over a static pressure of approximately 90 hPa, a pulsing pressure pattern appears which reflects the pilot's breathing. The respiration rate can be easily determined from the time scale in seconds and in this case is approximately 24 breaths per minute. The respiration pressure picture is superposed by slight movements both of the pilot and also of the aircraft. The former is reflected in rapid shifts, and the latter in slower shifts, of the oscillation zero point of the respiration pressure.

Since the volume of the suit is variable only to a very slight extent, inhalation causes a slight volume increase of the pilot, which is expressed in a rise of the hydrostatic liquid column and thus of the internal pressure of the suit.

FIG. 4 is a pressure/time diagram recorded during a flight maneuver with increased local z acceleration for approximately 40 seconds. Here too, the pressure variation caused by breathing is clearly visible. Using data processing methods known per se, such pressure/time functions can be processed and divided into the individual superposed functions such as z acceleration and pulse and individually assessed.

In particular, aspects such as correct fit, the pilot's breathing technique, and, if necessary, also more technical flight parameters can be assessed individually and in detail. Moreover, it is important for the pilot himself to be able to objectively assess the correct fit before take-off, for example based on pressure amplitude, and this is provided for and made possible by viewing the image on the display device. When flying high-performance aircraft with the ability to withstand tight radii of turn at high speeds, it is crucial that the pilot masters an appropriate breathing technique. This breathing technique is indicated in aviation medicine and is learnable. The view of the breathing pattern on the display device 6 serves as a learning aid.

Of course, the pressure measurement cell 3 can also be applied at another point on the suit, in a liquid-conveying vein 2, for example in the chest region.

However, if, as was described at the outset, the pressure measurement cell 3 is fitted at the lowest possible point of a vein 2, it can then serve at the same time as a measurement device for the local z acceleration. Moreover, the breathing pattern is then clearly distinguished from the acceleration-induced pressure, as can be seen from FIG. 4.

Of course, the use of the device according to the invention is also possible in an orthostasis suit, for example according to EP 0 986 356, or in what is called a hypoxia garment, for example according to Swiss patent application 1610/02, and may also be indicated on medical grounds.

In said hypoxia garment, the device for measuring respiration rate has no liquid-conveying veins and is thus pushed into a liquid-filled pocket under the elastically pretensioned skin of the garment and secured there by suitable means.

1. A device for measuring the respiration rate and the breathing pattern of a person wearing an anti-blackout suit operating according to the hydrostatic principle, with liquid-conveying veins (2) which can extend substantially the entire length of the anti-blackout suit, an orthostasis suit or a hypoxia garment, characterized in that
a pressure measurement cell (3) is present which is inside a liquid-filled, liquid-tight sheath and is in pressure-communicating connection with one of the veins (2), or with the inside of the garment,
an evaluation apparatus (4) is present which evaluates and processes the measurement values of the pressure measurement cell (3) and is set up in such a way that it can feed both a display device (6) and a memory device (7).

2. The device as claimed in patent claim 1, characterized in that the pressure measurement cell (3) is arranged in the inside of a vein (2) of an anti-blackout suit.

3. The device as claimed in patent claim 1, characterized in that the pressure measurement cell (3) is arranged in the inside of a vessel which is connected via a tube to a vein (2) of the anti-blackout suit in such a way that it communicates with this vein (2) in a liquid-communicating and pressure-communicating manner.

4. The device as claimed in patent claim 2 or 3, characterized in that the pressure measurement cell (3) is situated at the level of the lowest point of the vein (2) of the anti-blackout suit.

5. The device as claimed in patent claim 2 or 3, characterized in that the pressure measurement cell (3) is connected via a cable (5) to the evaluation apparatus (4), which cable (5) transmits to the evaluation apparatus (4) the values determined by the pressure measurement cell (3).

6. The device as claimed in patent claim 2 or 3, characterized in that an optocoupler is present and is linked to the pressure measurement cell (3), said optocoupler transmitting to the evaluation apparatus (4) the values determined by the pressure measurement cell (3).

7. The device as claimed in patent claim 2 or 3, characterized in that a radio apparatus is present and is linked to the pressure measurement cell (3), said radio apparatus transmitting to the evaluation apparatus (4) the values determined by the pressure measurement cell (3).

8. The device as claimed in patent claim 1, characterized in that a display device (6) is present and is linked to the evaluation apparatus (4).

9. The device as claimed in patent claim 1, characterized in that a memory device (7) is present and is linked to the evaluation apparatus (4).

10. The device as claimed in patent claim 1, characterized in that both a display device (6) and also a memory device (7) are present and both are linked to the evaluation apparatus (4).
